(19) United States
(12) Patent Application Publication
(10) Pub. No.: US 2023/0048205 A1
(43) Pub. Date: Feb. 16, 2023

(54) METHOD AND APPARATUS FOR STERILIZATION OF RESPIRATORY MASKS

(71) Applicant: O2 UNLIMITED LLC, West Palm Beach, FL (US)

(72) Inventors: Sven LEPSCHY, Palm Beach Gardens, FL (US); Aaron WILLIAMS, Kalamazoo, MI (US); Derek VANDERWEIDE, Grand Rapids, MI (US); Daniel OSTERHOUSE, Mattawan, MI (US); Thomas VAUGHN, Battle Creek, MI (US); Thomas MELTZ, Kalamazoo, MI (US); Jesse MANTYLA, Battle Creek, MI (US)

(21) Appl. No.: 17/975,225

(22) Filed: Oct. 27, 2022

Related U.S. Application Data
(63) Continuation of application No. PCT/US2021/029885, filed on Apr. 29, 2021.
(60) Provisional application No. 63/017,577, filed on Apr. 29, 2020.
(60) Provisional application No. 63/121,487, filed on Dec. 4, 2020.

(51) Int. Cl. A61L 2/24; A61L 2/10; A61L 2/20

(52) U.S. Cl. CPC A61L 2/24; A61L 2/10; A61L 2/208

(57) ABSTRACT

A sterilization apparatus for sterilizing a respiratory mask is described. The sterilization apparatus comprises a sterilization chamber, an articulating mask support disposed inside the sterilization chamber, a bi-directional fan system disposed inside the sterilization chamber and configured to move air through an air passage to simulate human breathing, and a sterilization agent generator coupled to the sterilization chamber. The bi-directional fan system pumps air that includes the sterilization agent back and forth through the oxygen mask via the air passage, to mimic normal breathing of an individual using the mask.

### CROSS-REFERENCE TO RELATED APPLICATIONS

This application is a Bypass Continuation Application of International Application No. PCT/US2021/029885, filed Apr. 29, 2021, which claims the benefit of U.S. Provisional Application No. 63/017,577, filed Apr. 29, 2020, and U.S. Provisional Application No. 63/121,487, filed Dec. 4, 2020. The entire disclosure of each of these applications is hereby incorporated herein in its entirety by reference.

### TECHNICAL FIELD

The present invention relates generally to sterilizing respiratory devices for transporting a gas to and from an airway of a user. More specifically, the invention relates to respiratory devices, such as masks, that permit individuals to avoid inhospitable environmental situations including low oxygen levels, noxious gases, particulates, and pathogens, and to sterilization of such masks.

### BACKGROUND

A variety of respiratory masks are known, which contact areas surrounding the nose and/or mouth of an individual to create an effective seal. This allows breathable gas at above, below, or equal to ambient pressure to be provided to the individual in inhospitable environmental situations including low amounts of oxygen, noxious gases, particulates, or pathogens such as viruses, bacterial, prions and parts thereof. Firefighters, for example, are routinely provided with oxygen systems which include a mask that protects the user's airway as well as eyes. Masks are also utilized in aviation applications to provide oxygen to pilots in emergency situations. Other applications include swimming, mining, and various medical applications such as bronchoscopy, esophagogastroduodenoscopy, and transesophageal echocardiograms. Such masks are also used in training devices for these widely divergent uses. Such masks are generally proffered to nasal cannula and provide protection from external pathogens such as bacteria, viruses, prions and noxious gases. Training devices, including flight simulations, often incorporate such oxygen and face masks.

Such masks are usually high quality products and very expensive, costing thousands of dollars per mask in aviation applications, for example, and are, therefore, made to be used multiple times instead of being of a single-use disposable nature. In many situations, the masks cannot be assigned to one particular individual, but instead, are often shared among multiple individuals due to their expense. Commercial airliners, for example, are equipped with oxygen masks that must be worn by one pilot when the other leaves the cockpit and the aircraft is above a prescribed altitude. The masks are not changed between crew changes on an aircraft. Aircraft simulators also include oxygen systems with masks to train pilots on procedures in the event of aircraft depressurization. These masks are repeatedly used by a number of individuals undergoing simulator training.

The use of respiratory masks by multiple people or at different times can create cross contamination from pathogens such as bacteria and viruses, from previous users of the masks. Past efforts to prevent cross-contamination include providing a disposable filter element within the mask that can be changed between users, using masks where a part or parts of the mask that were in contact with the initial user or the user's breath are disposable, wiping the mask with a sterilizing or sanitizing solution or, perhaps, a combination of two or more of these techniques. While such efforts may be sufficiently effective for pathogens that are not highly contagious, there is concern that conventional methods may not be satisfactory in situations in which highly contagious pathogens may have passed through the mask. This is particularly true for high-end oxygen masks that include valves that open and close as the user is breathing.

Moreover, these high-end oxygen masks often include sensitive electronics or pure oxygen supply lines and cannot be sanitized using sterilization agents that leave a residue. Further, the electronics are often not water-proof. Thus, conventional sterilization agents with chlorine-based sanitizers cannot be used to sterilize high-end oxygen masks.

Thus, there is continued need in the art for personal and/or disposable face or respiratory masks to avoid cross-contamination. There is also a continued need for improved methods and systems for sterilization of respiratory devices in a manner to prevent highly contagious pathogens to be passed between users.

### SUMMARY OF THE INVENTION

Some embodiments of the invention provide a method and apparatus for sterilization of respiratory devices in a manner to prevent highly contagious pathogens from being passed between users of the respiratory devices. In some embodiments, a sterilization apparatus is provided that includes a sterilization chamber in which an articulating mask support is located, a bi-directional fan system configured to move air through an air passage to simulate human breathing, and a sterilization agent generator coupled to the sterilization chamber. A control processor is provided to control operation of the articulating mask support, the bi-directional fan system, and the sterilization agent generator.

In some embodiments, the sterilization agent generator includes an ionized hydrogen peroxide generator system coupled to the sterilization chamber to provide a sterilization agent into the sterilization chamber. In some embodiments, the sterilization agent generator includes a sanitizing system that uses ultraviolet (UV) light to generate hydroxyls as a sterilization agent. The sanitizing system is coupled to the sterilization chamber to provide the sterilization agent into the sterilization chamber.

In some embodiments, a UV lighting system is provided within the sterilization chamber to provide a secondary sterilization agent.

In some embodiments, the articulating mask support functionally mimics the form of a human head such that an oxygen mask can be strapped to the articulating mask support in a manner consistent with normal use. In some embodiments, the articulating mask support is divided into a front face and back portion such that the front face and back portion are moveable with respect to one another.

In some embodiments, during operation of the sterilization apparatus, an operator places an oxygen mask on the articulating mask support and seals the sterilization chamber. The control processor sends a command signal to the articulating mask support causing the back portion of the articulating mask support to move relative to the front face of the articulating mask support in a first direction, thereby stretching straps of the oxygen mask to tighten the oxygen mask against the front face of the articulating mask support. Once a proper seal has been established between the oxygen mask and the front face, the command processor activates the sterilization agent generator to flood the sterilization chamber with a sterilization agent so that sealed portions of the mask are sterilized. The command processor also activates the bi-directional fan system to pump air back and forth through the oxygen mask via the air passage. The volume and pressure of air moved by the bi-directional fan system is set to mimic normal breathing of an individual using the oxygen mask. As the air passes back and forth through the oxygen mask, any diluter system and/or exhaust valves contained within the mask are exercised so that the sterilization agent reaches every potentially contaminated surface within the oxygen mask. While the sterilization agent should be sufficient to neutralize any highly infectious pathogens of concern, the UV sterilization system can also be activated to provide a further sterilization agent.

In some embodiments, the control processor is further configured to send a command signal to the articulating mask support to cause the back portion of the articulating mask support to move relative to the front face of the articulating mask support in a second direction opposite to the first direction, thereby loosening the mask against the front face of the articulating mask support such that the seal is broken, so that unsealed portions of the mask are sterilized.

Some embodiments of the invention provide respiratory masks that are disposable or can be sanitized for reuse. In some embodiments, the respiratory masks may include one or more of a quick disconnect kit, a form fit shield, or a stowage container. In some embodiments, the respiratory masks may be sanitized using one or more of certified or non-certified sterilizers, sterilization containers, pressurized gas, or UV lighting.

In some embodiments, the quick disconnect kit includes a fitting assembly installed into the gas hose line at any point along the line and facilitates the rapid connect and disconnect of a respiratory mask. In some embodiments, the respiratory mask may include a one-way valve prohibiting bacteria, viruses, or other pathogens from entry upstream of the disconnect kit in the gas hose line.

In some embodiments, the form fit shield includes a combination of ventilator material and vent that is form fit around or inside of the seal to prevent particulates from reaching the recesses of the masks.

In some embodiments, the respiratory mask may be placed in a stowage container that sanitizes and protects the mask. In some embodiments, the stowage container may include an insert that allows routing of the tubing and wiring necessary for the use of the mask. In some embodiments, the stowage container may include an ultraviolet (UV) lighting system for sterilizing the mask. In some embodiments, a sanitizing system may distribute a sanitizing spray or pressurized gas through the mask and tubing to sterilize the same.

In some embodiments, a protective sleeve is wrapped around a sanitized mask to protect the mask from any contamination after sterilizing.

In some embodiments, a sterilization system utilizes pure oxygen under pressure, or other sanitizing gases such as ozone or nitrous oxide (NO) to penetrate the crevasses and provide sanitation of mask and tubing areas the gas touches. In some embodiments, the sterilization system includes a nozzle to distribute the sanitizing gas.

In some embodiments, the mask may include a filter to remove pathogen material that is exhaled.

In some embodiments, the mask may include electronic components such as earphones or microphones. In some embodiments, the electronic components may be waterproof, water-resistant, or removable to mitigate damage from liquids or sanitizing sprays.

In some embodiments, a sterilization system utilizes heat, steam, or an autoclave to destroy pathogens in the mask. In some embodiments, various pasts of the mask may be made with heat-resistant materials.

### BRIEF DESCRIPTION OF DRAWINGS

It is to be understood that the attached drawings are for purposes of illustrating aspects of various embodiments and may include elements that are not to scale.

FIG. 1 shows a respiratory mask, according to embodiments of the invention.

FIG. 2 shows a respiratory mask with a filter, according to embodiments of the invention.

FIG. 3 shows a respiratory mask with a form fit shield, according to embodiments of the invention.

FIG. 4 shows sterilized or sanitized parts of a respiratory mask, according to embodiments of the invention.

FIG. 5 shows a stowage container with optional sterilizing unit and protection unit for a respiratory mask, according to embodiments of the invention.

FIG. 6 shows another stowage container for a respiratory mask, according to embodiments of the invention.

FIG. 7 shows a pressurized gas supply for sterilizing a respiratory mask, according to embodiments of the invention.

FIG. 8 shows ultraviolet lighting source for sterilizing a respiratory mask, according to embodiments of the invention.

FIG. 9 shows an exhaust filter for removing exhaled air in a respiratory mask, according to embodiments of the invention.

FIG. 10 shows a respiratory mask sterilizing and stowage system, according to embodiments of the invention.

FIG. 11 shows a respiratory mask with waterproof electronic components, according to embodiments of the invention.

FIG. 12 shows a respiratory mask with removable electronic components, according to embodiments of the invention.

FIG. 13 shows a mask for virtual or augmented reality applications, according to embodiments of the invention.

FIG. 14 shows a sterilizing apparatus for a respiratory mask, according to embodiments of the invention.

### DETAILED DESCRIPTION OF THE INVENTION

The invention will be described with reference to certain preferred embodiments and the accompanying figures which show various embodiments of a respiratory mask, sterilization apparatus, and stowage devices, in accordance with the invention. It will be understood, however, that the scope of the invention is not limited to those specifically disclosed embodiments. As just one example, aviation respiratory masks and their sterilization will be described below, but the invention is applicable to respiratory masks and devices other than those used in aviation.

In this regard, in the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

The word “sterilizing”, the word “sanitizing”, and the word “cleaning” are used interchangeably in the description and are all intended to convey a process that removes, kills, or deactivates various forms of life (in particular referring to microorganisms such as viruses, bacteria, fungi, and spores).

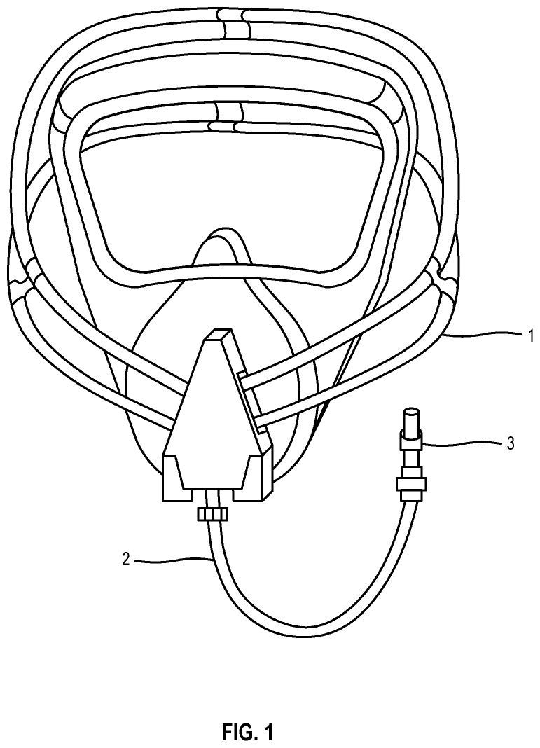

FIG. 1 shows a respiratory mask 1 (also referred to as mask or respiratory device) according to an embodiment of the invention. The respiratory mask 1 may be a personal disposable mask or a personal individual mask. A personal disposable mask is generally used for personnel undergoing training. In some embodiments of the invention, the personal disposable mask 1 is fabricated from economical materials that support a sterilization method for multiple uses. Such materials include but are not limited to silicon plastic, rubber, or cloth.

A personal individual mask preferably stays with the user, e.g., pilot, while in training and when assigned for duty in a duty station, e.g., aircraft. In some embodiments, the respiratory mask 1 is compatible with a quick disconnect kit 3 (also referred to as a quick connect kit or a quick disconnect/connect kit). In some embodiments, the quick disconnect kit 3 connects the respiratory mask to an air supply. In some embodiments, the respiratory mask 1 may include one or more of the accessory solutions described in this application.

In some embodiments, the quick disconnect kit 3 includes a fitting assembly installed into the gas (e.g. oxygen) hose line 2 at any point along the line 2. In some embodiments, the quick disconnect kit 3 facilitates the rapid connect and disconnect of a respiratory mask 1 such as an oxygen mask to an oxygen supply.

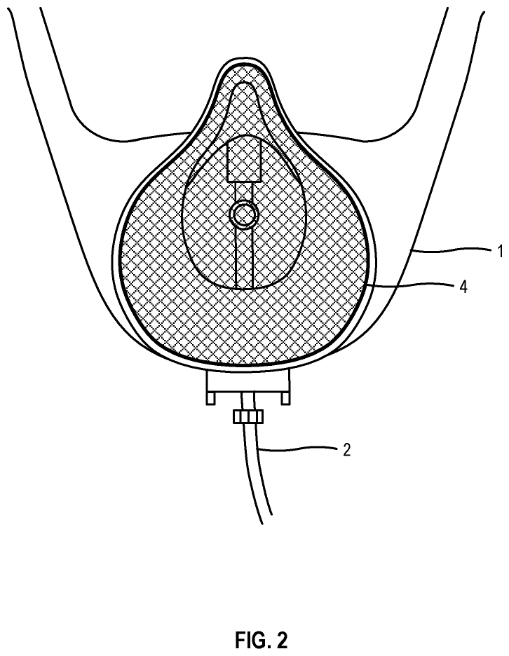

In some embodiments, as shown in FIG. 2, the mask 1 may include a one-way valve or filter 4 prohibiting bacteria, viruses, or other pathogens from entry upstream of the quick disconnect kit 3 in the gas line 2. In some embodiments, the filter 4 permits flow of gas in one direction but not the other.

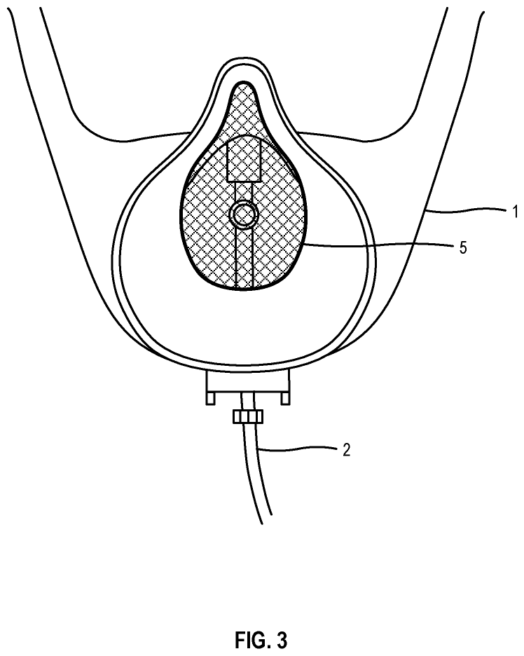

FIG. 3 shows a respiratory mask 1 including a form fit shield 5, according to some embodiments of the invention. In some embodiments, the form fit shield 5 may be any kind of ventilator material and vent combination, (e.g. similar to a N95 mask) that is form fit around or inside of the seal to prevent particulates from reaching the recesses of the mask 1.

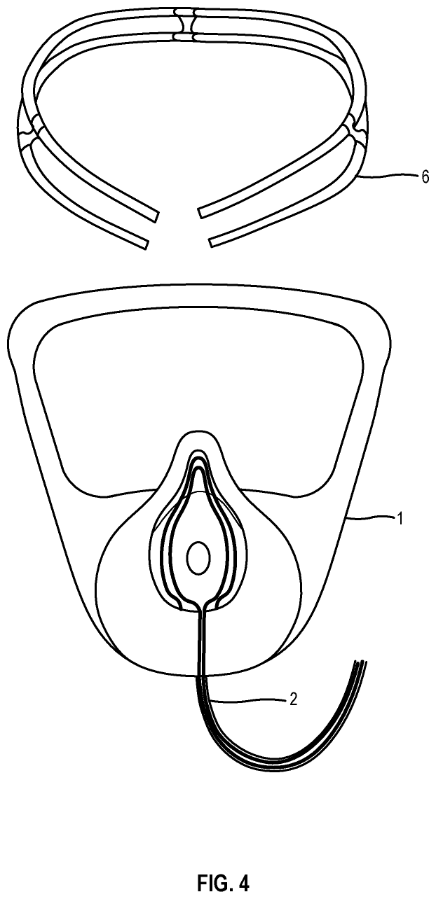

In some embodiments, as shown in FIG. 4, the mask 1 and hose line (tubing) 2 may be cleaned, sanitized, or sterilized with any method of sterilization. In some embodiments, the straps 6 may be separated from the tubing 2 or other portions of the mask 1 for separate sterilizing or disposal. In some embodiments, the mask 1 and tubing 2 may be sterilized by distributing a sanitizing gas through the tubing 2 or mask 1. In some embodiments, the mask 1 and tubing 2 may be sterilized by radiating the tubing 2 or mask 1 with ultraviolet (UV) light.

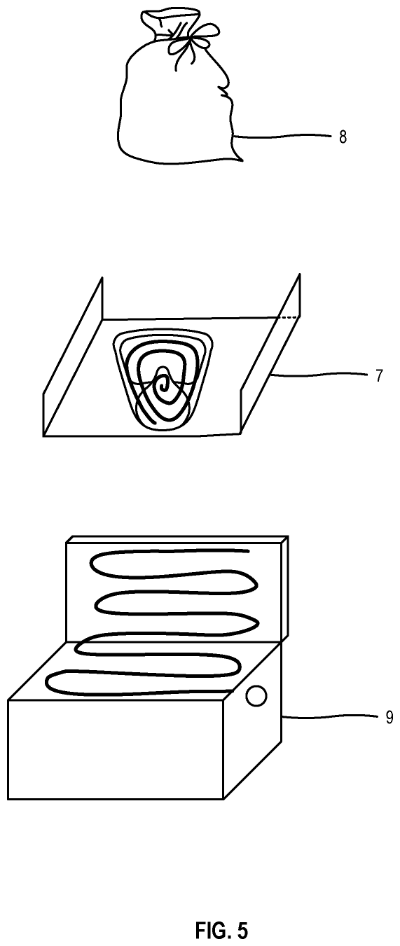

FIG. 5 shows a certified stowage container 9 for the mask 1, according to some embodiments of the invention. Certification may be provided by approval of the certified stowage container 9 by a relevant authority, which may include the Federal Aviation Administration (FAA) or the Food and Drug Administration (FDA), amongst others. In some embodiments, the certified stowage container 9 may be modified by various methods of sterilizing the mask 1 or the tubing 2. In this regard, the stowage container 9 may include one or more of accessories such as a sterilization unit 7 or a protective sleeve 8, as described in this application.

FIG. 5 also shows a sterilization unit or insert 7, according to some embodiments of the invention. As depicted in FIG. 5, the insert may be designed such that it allows routing of the tubing 2 or wiring necessary for the use of the mask 1. In some embodiments, the insert 7 accommodates the mask 1 and tubing 2 to clean, sanitize, or sterilize the mask 1 or tubing 2 using any of the exemplar sterilization methods discussed in this application. In some embodiments, the sterilized mask 1 and tubing are stored within the insert 7, which may be place inside the certified stowage container 9

FIG. 5 also shows a protective sleeve 8 that is designed to wrap around a sanitized mask 1. The protective sleeve 8 protects the mask 1 from any contamination after it has been sterilized and is stored in the certified stowage container 9. In some embodiments, the protective sleeve 8 may seal the mask to protect against contamination.

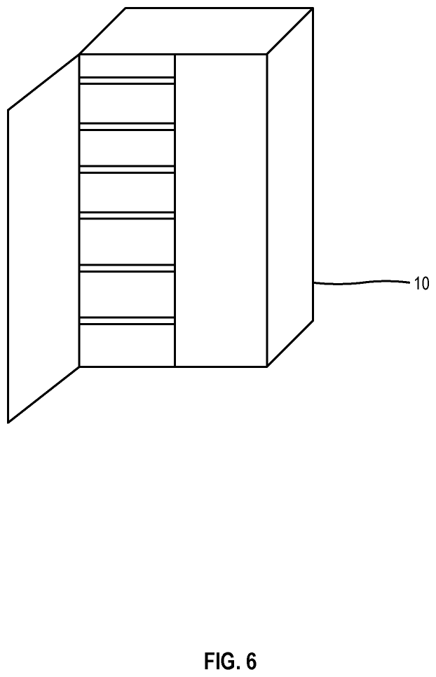

FIG. 6 shows a non-certified stowage container 10 for the mask 1, according to some embodiments of the invention. In some embodiments, the non-certified stowage container 10 may be a storage cabinet, storage box, or storage bucket. In some embodiments, the mask 1 may be cleaned or sterilized using before storage in the non-certified stowage container 10. In some embodiments, the cleaned or sanitized mask 1 may be placed in a protective sleeve 8 before storage in the non-certified stowage container 10.

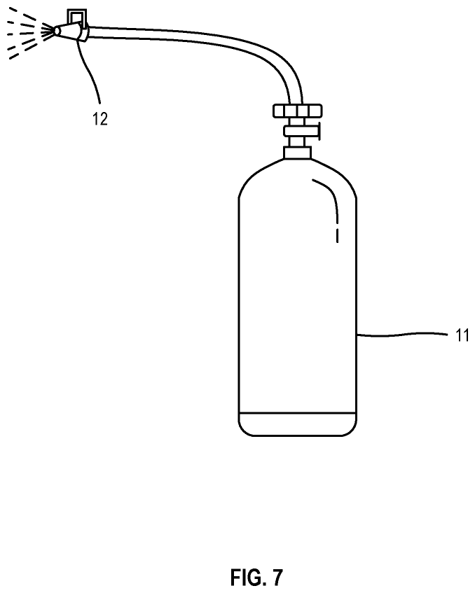

FIG. 7 shows an example of a pressurized gas supply 11 for sterilizing the mask 1, according to some embodiments of the invention. In some embodiments, the pressurized gas supply 11 includes a quick disconnect kit (also referred to as quick connect kit or quick disconnect/connect kit) 12 that couples with the quick disconnect 2 kit 2 provided on mask 1 to permit pressurized gas to be distributing through the mask 1 and tubing 2 for sterilization.

In some embodiments, the pressurized gas includes pure oxygen under pressure or any other sanitizing gas such as ozone or nitrous oxide (NO) that penetrates the crevasses of the mask 1 and provides sanitation of areas the gas touches.

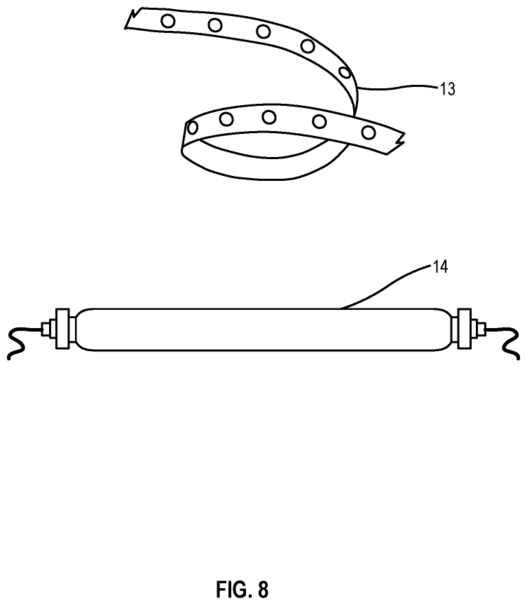

Sterilization of the mask is not limited to methods that require distribution of a sanitizing gas and may also be performed using ultraviolet light strips 13 or bulbs 14, as shown in FIG. 8. In some embodiments, the UV light is in the UV-C wavelength range of 100-280 nm, well known in the art to provide germicidal properties. In some embodiments, the certified stowage unit 9 or the non-certified stowage unit 10 includes a UV device that distributes UV lighting within the stowage unit that holds the mask or to expose the entire stowage unit. In some embodiments, the UV device includes a timer that turns-off the UV light source after a predetermined time to render any pathogens safe and limit UV damage to the mask 1.

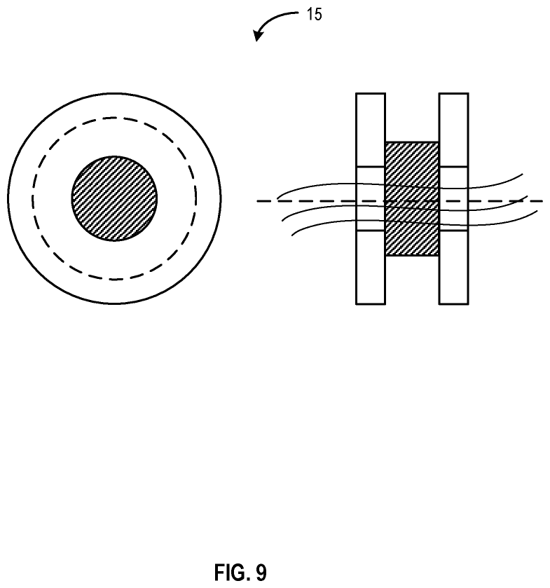

In some embodiments, the mask 1 may be maintained in a cleaned or sanitized state using a filter 15, as shown in FIG. 9, to remove pathogen material from air exhaled by a user of the mask 1. The filer 15 prevents the exhaled pathogens from entering the tubing 2 or other sealed areas of the mask 1, requiring only a sterilization of the exhalation area of the mask. In some embodiments, a sanitation spray may be distributed within the confines of the container containing the mask 1. Such a sanitizing solution could contain, without limitation, detergent solutions, hydrogen peroxide solutions, alcohol solutions, ozone gas, etc. This embodiment can be used on the mask 1 or on its container 9 or 10, and can be used with any other embodiments for cleaning and sanitizing the mask 1 described in this application.

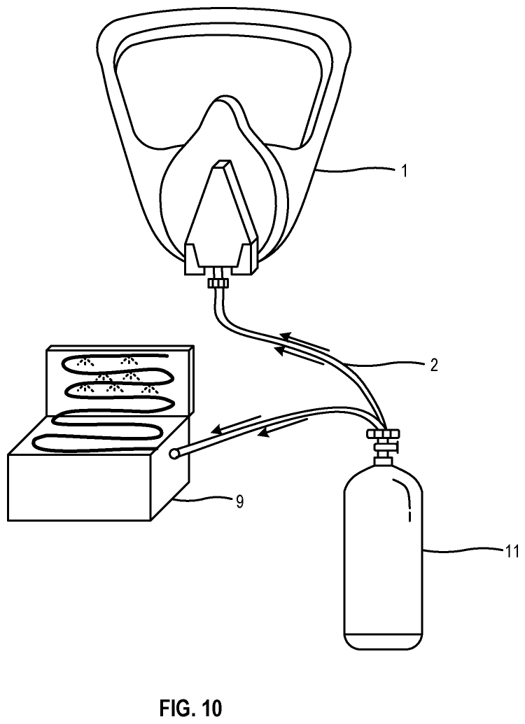

FIG. 10 shows a system for sterilizing and stowing a mask 1, according to some embodiments. The mask sterilization and stowing system may include a stowage container, such as the certified stowage container 9, which stows the mask 1 when the mask is not in use. In some embodiments, a pressurized gas sanitation system 12 may be connected to the stowage container to sterilize the stored masks. In some embodiments, a pressurized gas sanitation system 12 may be connected to the mask 1 via tubing 2 to directly sterilize the mask.

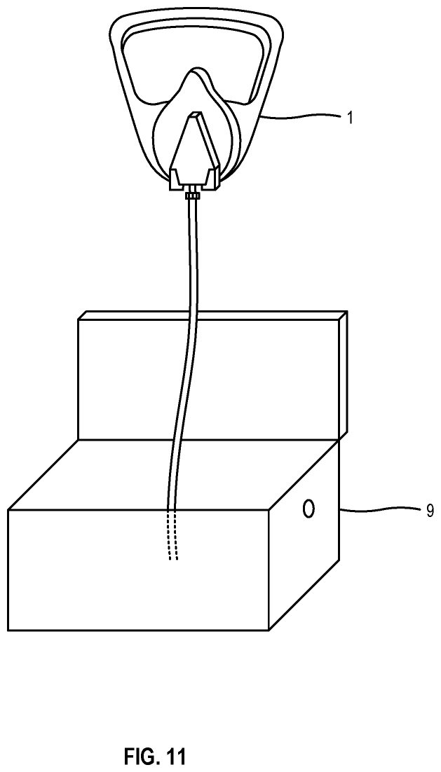

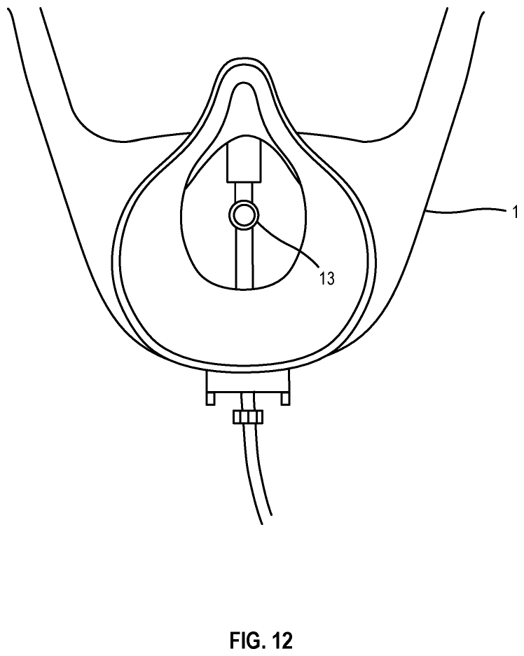

In some embodiments, the mask 1 may include electronic components 13 such as a microphone or earbuds, as shown in FIG. 12. In some embodiments, as shown in FIG. 11, the electronic components 13 are waterproof so that they are not damaged by sanitizing sprays used to sterilize the mask, such as, for example, when the mask 1 is stored in the stowage container 9. In some embodiments, the electronic components 13 are removable from the mask 1 so that they are not damaged by sanitizing sprays used to sterilize the mask, such as, for example, when the mask 1 is stored in the stowage container 9. In some embodiments, Bluetooth technology may be used to connect the electronic components 13 to make the mask either waterproof or water resistant and mitigate damage from liquids, sanitizing mists, or high temperatures. These embodiments can be combined with any of the embodiments set forth in this application.

Embodiments of the invention are also contemplated that include using materials that can be heat treated, steam treated or autoclaved to destroy pathogens, permitting the use or safe disposal of masks 1. These embodiments can be combined with one or more embodiments set forth in this application.

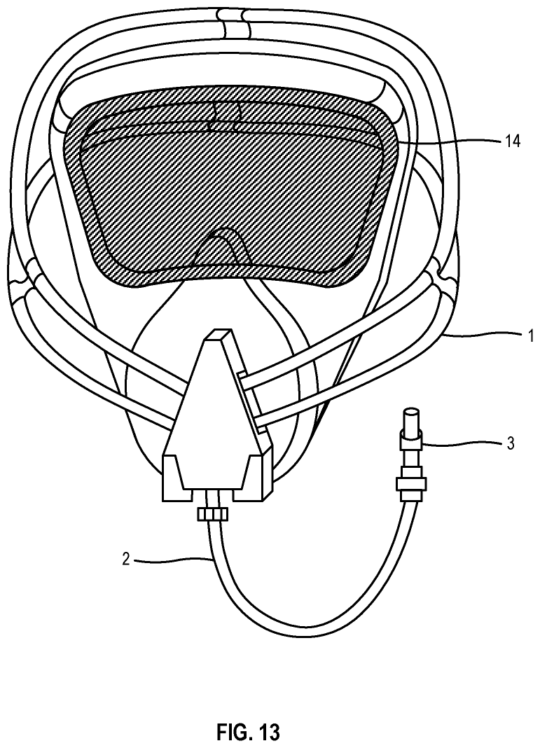

FIG. 13 shows an embodiment of a mask 1 used in virtual or augmented reality applications. In these embodiments, the mask 1 may also include a display 14 to permit the user to interact with the virtual or augmented environment. Virtual Reality and Augmented Reality Software and Hardware comprise embodiments that allow for a disposable personal mask attachment. This can also be useful for e-gaming to increase the realism of the experience. Such a mask is useful for oxygen training and evaluation. These features can be used with one or more embodiments set forth in this application.

In some embodiments, various sanitation or sterilizing methods may be used to sterilize the mask 1. It should be noted that the invention is not limited to the details of these sanitation methods, which are provided for purposes of illustration only. Some examples of sanitation methods include heat, alcohol solutions, alcohol mists, hydrogen peroxide solutions, hydrogen peroxide mists, ozone gas, pure oxygen gas, steam, UV lighting, detergent solutions and mists, ammonia solutions, perchlorate or bleach solutions.

In some embodiments, very high heat that kills pathogens may be used to disinfect the mask 1. In some embodiments, the mask or parts of the mask to be disinfected may be placed in an autoclave or have heat applied to any surface intended to be sanitized.

In some embodiments, the mask 1 may be sterilized by using a high percent alcohol solution of at least 70% alcohol to disinfect the mask. In some embodiments, the mask or parts of the mask to be disinfected may be fully immersed into a solution to apply alcohol to any surface intended to be sanitized.

In some embodiments, the mask 1 may be sterilized by misting or spraying the mask with a high percent alcohol solution of at least 70% alcohol. In some embodiments, the mask or parts of the mask to be disinfected may be fully sprayed by a solution to apply alcohol to any surface intended to be sanitized.

In some embodiments, the mask 1 may be sterilized by dipping the mask into a hydrogen peroxide solution. In some embodiments, the mask or parts of the mask to be disinfected may be fully immersed into a solution to apply hydrogen peroxide to any surface intended to be sanitized.

In some embodiments, the mask 1 may be sterilized by misting or spraying the mask with a hydrogen peroxide solution. In some embodiments, the mask or parts of the mask to be disinfected may be fully sprayed by a solution to apply hydrogen peroxide to any surface intended to be sanitized.

In some embodiments, the mask 1 may be sterilized by using isolated ozone gas to disinfect the mask. In some embodiments, the mask or parts of the mask to be disinfected may have ozone gas applied to any surface intended to be sanitized. The method of application may include pressurized gas or immersing the mask into a chamber that does or will contain ozone gas.

In some embodiments, the mask 1 may be sterilized by using isolated pure oxygen gas to disinfect the mask. In some embodiments, the mask or parts of the mask to be disinfected may have pure oxygen gas applied to any surface intended to be sanitized. The method of application may include pressurized gas or immersing the mask into a chamber that does or will contain pure oxygen gas.

In some embodiments, the mask 1 may be sterilized by using heated steam to disinfect the mask. In some embodiments, the steam is applied to any mask surface intended to be sanitized. Alternatively, in some embodiments, the mask or parts of the mask may be immersed into a chamber that does or will contain heated steam. In some embodiments, the steam shall be hot enough to sanitize any surface. In some embodiments, the steam may be pressurized.

In some embodiments, the mask 1 may be sterilized by using UV-C lighting to disinfect the mask. In some embodiments, the mask or parts of the mask to be disinfected may have UV-C light applied to any surface intended to be sanitized. The method of dispersing UV-C may vary and may include photoactivated reactive molecules.

In some embodiments, the mask 1 may be sterilized by using detergent solutions, e.g., sodium lauryl sulfate. Detergent solutions are well known in the art to dissolve the lipid envelopes of viruses and disrupt bacteria, thereby removing potential pathogens. In some embodiments, ammonia and its salts can also be used to help sanitize the surfaces of the mask and its containers.

In some embodiments, the mask 1 may be sterilized by using ionized hydrogen peroxide as a sterilizing agent. In some embodiments, that mask 1 may be sterilized by using ultraviolet light to convert moisture in air into hydroxyls and organic oxidants that act as sterilization agents. Hydroxyls and organic oxidants kill airborne viruses, bacteria, and mold. Hydroxyls react with volatile organic compounds (VOCs) to neutralize and decompose these chemicals and to generate powerful organic oxidants, which also sanitize but are not as reactive so they exist longer than hydroxyls. Hydroxyls and organic oxidants do not negatively impact materials, making them safe for use in high-end oxygen masks. Hydroxyls and organic oxidants also eliminate cigarette smoke, chemical odors, and mildew, providing additional benefits beyond sterilization.

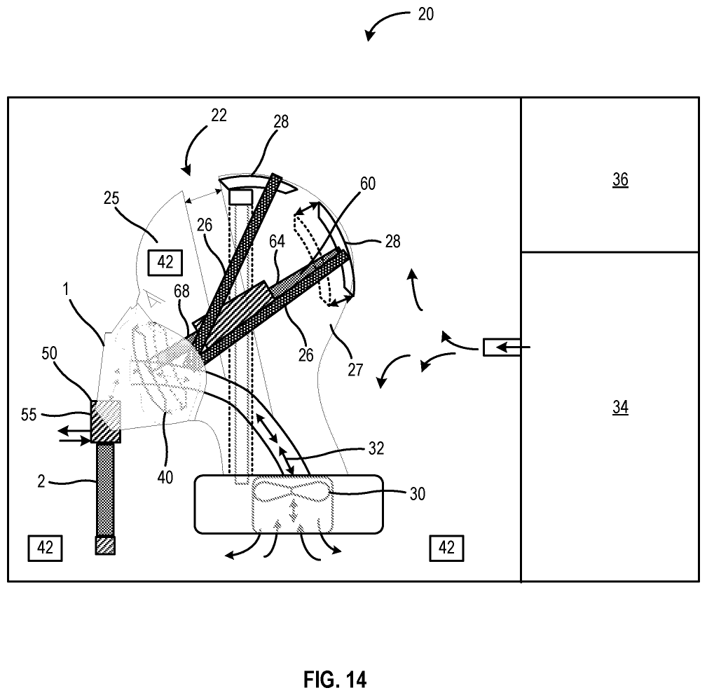

FIG. 14 shows a sterilization apparatus for sterilizing a respiratory device, according to some embodiments of the invention. The sterilization apparatus includes a sterilization chamber 20 having an articulating mask support 22, a bi-directional fan system 30 that is capable of moving air through an air passage 32 to and from an oxygen mask 1 to be placed on the mask support, a sanitizing system 34 for generating a sterilization agent (for example, an ionized hydrogen peroxide generator or a hydroxyl and organic oxidant based sanitization system), and a control processor 36 for controlling the overall operation of the apparatus.

In the illustrated example shown in FIG. 14, the articulating mask support 22 preferably takes the form of a human head mannequin so that a mask 1 (such as an oxygen mask) can be strapped to the articulating mask support 22 in a manner consistent with normal use. It should be noted that the articulating mask support 22 only needs to mimic certain functional aspects of a human head that relate to placing or positioning a mask similar to how the mask would be used by a human, and may not resemble a human head in all physical aspects. In some embodiments, the oxygen mask 1 includes straps 26 that are used to secure the oxygen mask 1 onto a user's head.

In the case of aviation masks used in a diluter or demand system 50 for high altitude flight, the straps 26 usually are inflatable to permit the oxygen mask 1 to be easily placed around a user's head. The straps are then deflated so that the mask 1 is held tight against the user's face in order to provide a proper seal. In such cases, in some embodiments, it is not desirable to inflate the straps 26 during a routine sterilization process. Accordingly, another method of making sure that the oxygen mask 1 is secured tight against the articulating mask support 22 (i.e. the face of the human head mannequin) may be used.

In the embodiment shown in FIG. 14, top and back strap rests 28 are provided at the top and back of the articulating mask support 22. The articulating mask support 22 is also divided into a front face 25 and back portion 27 such that the front face 25 and back portion 27 are moveable with respect to one another in a forward direction (increasing separation between the front face 25 and back portion 27) and a backward direction (decreasing separation between the front face 25 and back portion 27). In some embodiments, the front face 25 and back portion 27 may be hinged together or spring loaded to allow relative motion between the two faces. In some embodiments, the front face 25 and back portion 27 by be two separate moveable elements located proximate to one another. In some embodiments, an actuator 60 is provided that includes an actuator arm 64 that pushes against the back portion 27 of the mask support 12 and an actuator arm 68 that pushes against a mask rest 40 provided on the front face 25. The actuator 60 can be either an electrically or pneumatically operated actuator and multiple actuator devices may be employed as opposed to one device with multiple arms. In some embodiments, the mask rest 40 is moveable with respect to the front face 25.

Operation of the sterilization apparatus, according to some embodiments of the invention, will now be described. In some embodiments, an operator places the oxygen mask 1 on the articulating mask support 22 and closes the sterilization chamber 20. Upon activation by a user control, or alternatively responding automatically to the closing of the sterilization chamber 20 through a switching device, the control processor 36 sends a command signal to the actuator 60 to activate the actuator arm 64. As the actuator arm 64 extends, the back portion 27 of the mask support 22 moves relative to the front face 25 to increase the separation between the front face 25 and back portion 27, thereby stretching the straps 26 and tightening the oxygen mask 1 against the front face 25 of the mask support 22. Once a proper seal has been established, the command processor 36 activates the sanitizing system 34 to flood the sterilization chamber 20 with a sterilization agent such as hydrogen peroxide vapor or UV treated air having hydroxyls and organic oxidants. In some embodiments, either before or after the activation of the sanitizing system 34, the command processor 36 activates the bi-directional fan system 30 to pump air back and forth through the oxygen mask 1 via the air passage 32. The volume and pressure of air moved by the bi-directional fan system 30 is set to mimic normal breathing of an individual using the oxygen mask 1. As the air passes back and forth through the oxygen mask 1, any diluter system 50 and/or exhaust valves 55 contained within the mask are exercised so that the sterilization agent reaches every surface within the oxygen mask 1.

In other words, the mannequin (mask support 22) and bi-directional fan system 30 essentially mimic the human breathing process of inhaling and exhaling air, causing the air flow to move in both directions. This simulated breathing process permits each valve in the mask 1 and tubing 2 to cycle through its different states (such as open or close), thereby fully sterilizing each valve.

In some embodiments, the tubing 2 may be effectively sealed from the other portions of the mask 1 by maintaining a positive pressure in the tubing 2. The positive pressure prevents contaminants from outside the tubing 2 to be drawn into the tubing. Thus, in these embodiments, the sterilization agent may not need to be diffused through the tubing 2.

In some embodiments, gas, such as air, is radiated with UV-C light to generate sterilization agents such as hydroxyls and organic oxidants, which are then moved back and forth across the valves. This eliminates the need for a specialized sanitizing gas such as hydrogen peroxide vapor to be used.

In some embodiments, during this process of bi-directionally moving the sterilization agent, the command processor 36 also sends a command signal to the actuator 60 to move the actuator arm 68 and push the mask rest 40 outward, thereby lifting the oxygen mask 1 slightly from the front face 25, thereby allowing the sterilization agent to also act on the surface of the oxygen mask 1 normally sealed against the front face 25. Once the sterilization process is complete, the sterilization agent is preferably vented from the sterilization chamber 20 through a carbon layer filter containing titanium dioxide.

While the sterilization agent should be sufficient to neutralize any highly infectious pathogens of concern, in some embodiments, the sterilization chamber 10 is also provided with UV lights 42 (preferably UV-C) positioned around the articulating mask support 22 to provide a further sterilization agent. In some embodiments, a UV light 42 is provided within the mask support 22 and the mask support 22 is made out of a material transparent to UV light, thereby providing UV light sterilization to the inside of the oxygen mask 1 as well as the outside surface thereof.

The invention has been described with reference to certain embodiments thereof. It will be understood, however, that modification and variations are possible. For example, operation of the sterilization apparatus can be through manual controls thereby eliminating the need for the control processor 36. Different sanitizing agents other than hydrogen peroxide or UV-treated air having hydroxyls or organic oxidants may also be employed. While the preferred mask support 12 takes the form of a human head mannequin, other structures can be employed to hold the oxygen mask 14 without specifically mimicking the human head, as long as the portion of the mask support 12 directly supporting the oxygen mask 14 provides a sufficient seal to mimic use of the oxygen mask 14 is normal operation. Still further, the embodiment illustrated in FIG. 14 uses the actuator 22 to move the two pieces of the articulating mask support 12 apart to stretch the straps of the oxygen mask 14 to provide a seal. This method is utilized as the oxygen masks 14 for aviation use normally use straps that are inflated with the aircraft oxygen supply in order to place the mask around the user's head and then deflated to tighten the mask around the user's face. The use of the articulating mask support 12 eliminates the need to incorporate a pressurized oxygen supply to the sterilization chamber 10, thereby reducing the complexity of the device while allowing the oxygen mask to be easily slipped on the articulating mask support 12 by an operator. However, if so desired, the mask straps could be inflated to place them around the mask support 22 and then deflated to tighten and seal, instead of using the actuator to move the split pieces of the articulating mask support These are but a few examples of possible variations and are not meant to limit the scope of the invention to these specific examples.

Subsets or combinations of various embodiments described above provide further embodiments.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

1. A sterilization apparatus comprising:
a sterilization chamber;
an articulating mask support disposed inside the sterilization chamber;
a bi-directional fan system disposed inside the sterilization chamber and configured to move air through an air passage to simulate human breathing; and
a sterilization agent generator coupled to the sterilization chamber.

2. The sterilization apparatus according to claim 1, further including a control processor configured to control operation of the articulating mask support, the bi-directional fan system, and the sterilization agent generator.

3. The sterilization apparatus according to claim 1,
wherein the sterilization agent generator includes a sanitizing system that uses ultraviolet (UV) light to generate hydroxyls as a sterilization agent, and
wherein the sanitizing system is coupled to the sterilization chamber to provide the sterilization agent into the sterilization chamber.

4. The sterilization apparatus according to claim 1, wherein the articulating mask support functionally mimics the form of a human head such that a mask can be strapped to the articulating mask support.

5. The sterilization apparatus according to claim 4, wherein the articulating mask support is divided into a front face and back portion such that the front face and back portion are moveable with respect to one another.

6. The sterilization apparatus according to claim 1, further including an ultraviolet (UV) lighting system disposed within the sterilization chamber to provide a secondary sterilization agent.

7. The sterilization apparatus according to claim 5, wherein the control processor is further configured to send a command signal to the articulating mask support to cause the back portion of the articulating mask support to move relative to the front face of the articulating mask support in a first direction, thereby stretching straps of the mask to tighten the mask against the front face of the articulating mask support such that a seal is formed.

8. The sterilization apparatus according to claim 7, wherein the control processor is further configured to, after the mask is tightened against the front face of the articulating mask support, activate the sterilization agent generator to flood the sterilization chamber with a sterilization agent.

9. The sterilization apparatus according to claim 8,
wherein the control processor is further configured to activate the bi-directional fan system to pump air back and forth through the mask via the air passage, and
wherein a volume and a pressure of the air moved by the bi-directional fan system is set to mimic normal breathing of an individual using the mask.

10. The sterilization apparatus according to claim 7,
wherein the control processor is further configured to send a command signal to the articulating mask support to cause the back portion of the articulating mask support to move relative to the front face of the articulating mask support in a second direction opposite to the first direction, thereby loosening the mask against the front face of the articulating mask support so that a seal portion of the mask is sterilized.

11. The sterilization apparatus according to claim 1,
wherein the sterilization agent generator includes a sanitizing system that uses ionized hydrogen peroxide as a sterilization agent, and
wherein the sanitizing system is coupled to the sterilization chamber to provide the sterilization agent into the sterilization chamber.
